(19) United States
(12) Patent Application Publication
(10) Pub. No.: US 2008/0131319 A1
(43) Pub. Date: Jun. 5, 2008

(54) Method of Real-Time/Inline Detection of Ultratrace Metallic Element Contained in Sample Liquid and Apparatus Therefor

(75) Inventors: Sayoko Haji, Okayama (JP); Masayuki Suzuki, Okayama (JP); Tadashi Saito, Tokyo (JP)

Correspondence Address: FRISHAUF, HOLTZ, GOODMAN & CHICK, PC, 220 Fifth Avenue, 16TH Floor, NEW YORK, NY, 10001-7708, US

(73) Assignee: FIAMO CORPORATION, Shinbuya-ku, Tokyo (JP)

(21) Appl. No.: 11/667,155

(22) PCT Filed: Nov. 4, 2005
(86) PCT No.: PCT/JP2005/020275

§ 371 Date: Jul. 10, 2007

(30) Foreign Application Priority Data
Nov. 4, 2004 (JP) 2004-321191

(51) Int. Cl. B01L 1/00; G01N 33/20

(52) U.S. Cl. 422/68.1; 422/102; 73/61.42

(57) ABSTRACT

[Problems] In a method of real-time/inline detection of an ultratrace metallic element contained in a sample liquid, to provide a means for enabling detection based on new combinations of samples and pretreatment liquids, which has not been possible heretofore at a high sensitivity.

[Means for Solving] A method of real-time/inline detection of an ultratrace metallic element contained in a sample liquid, comprising a sampling step of sampling from the sample liquid, a pretreatment step of treating the sample with a pretreatment liquid, and an analyzing step of analyzing the metallic element in a treated liquid that has passed through the pretreatment step, wherein the sample, the pretreatment liquid and/or the treated liquid are cooled, if an exothermic reaction is involved in the pretreatment step.

### TECHNICAL FIELD

The present invention relates to methods of real-time/inline detection of ultratrace metallic elements contained in sample liquids based mainly on sulfuric acid, nitric acid, hydrochloric acid, hydrofluoric acid, phosphoric acid, aqueous ammonia and the like, for example, that tend to generate heat through neutralization reaction and the like and apparatuses therefor.

### BACKGROUND ART

In recent years, importance of quick analyses at a site of sampling (on-site analysis) has been recognized. In the field of environment, for example, various problems at the global scale have become serious, such as global warming, ozone layer depletion, acid rains, aerial pollution and marine pollution that are eliciting themselves. In order to solve such problems, it is necessary to have a picture of precise realities, such as forms and quantities of existence of causative agents responsible for such environmental problems, for which it is essential that reliable on-site techniques be developed for analyzing trace elements.

Also, in semiconductor manufacturing processes, a variety of liquid chemicals are used for processes of cleaning Si wafers and the like, of exposure and development and of etching. When such liquid chemicals are contaminated with metallic impurities, product performance and yields may seriously and adversely be affected. In general, for semiconductor manufacturing processes, chemicals and/or water of extreme purities are used and, for the quality control of such chemicals, a solution for on-site analytical techniques for trace elements is indispensable.

For conventional analyses of trace metallic elements in semiconductor manufacturing processes, samples were collected for each liquid chemical and then treatment for increasing the detection sensitivity was made in a remotely located laboratory and the like according to a method applicable only to batch processes, such as enrichment, for which a highly sensitive analytical method such as inductively coupled plasma-mass spectrometer (ICP-MS) was relied upon. For such a method, however, treatment such as enrichment of samples was needed and, for that, at least one day was required to provide analytical results. Consequently, if a liquid chemical was determined as highly contaminated with impurities, all products associated with that liquid were wastefully disposed of, resulting in a decrease in yield.

In addition, as a procedure for improving the lower limit of detection, so called sensitization, a method has generally been known in which elements to be detected in a sample are enriched to derive the elemental concentration in the sample, taking the enrichment ratio into account. As methods for enrichment, those of performing evaporation and distillation in a vessel which is less contaminated with impurities, such as one made of platinum and/or synthetic quartz as well as those of adsorbing element components onto an adsorbent or collector, such as an ion exchange resin, for enrichment are in general practice. These methods must, however, be based on batch processing and, therefore, are not easily applicable to on-site analyses. Even if they are applicable to on-site analyses, they are still not applicable to analyses of ultratrace amounts because contamination from an ion exchange resin, concentrator, collector, even eluent and the like cannot be eliminated.

Under such circumstances, the inventors have proposed an on-site microanalysis with the application of flow injection analysis (FIA) as disclosed in Patent Reference 1. FIA is a method for analyzing elemental concentrations wherein a carrier (sample-carrying fluid) is flowed through a flowpath, replacing the carrier on a timely basis with a sample to be analyzed, so that the sample may react with a reaction reagent with which elements to be detected will develop colors, and the difference in absorbance between the carrier and the sample to be analyzed, A, is detected to analyze the elemental concentrations. In other words, in FIA, a carrier and a reaction reagent are mixed and thoroughly stirred by means of agitation and/or dispersion before detecting concentrations using a detector for detecting elemental concentrations (typically, determining absorbance by absorbance analyses) and, at a certain point of time, the carrier is replaced with a sample thereby determining the differential in absorbance to determine the sample concentration. The disclosure of Patent Reference 1 is in its entirety to be incorporated herein.

The principle of FIA will now be shown in FIGS. 1 and 2. With reference to FIG. 1, a carrier and a reaction reagent are constantly mixed and agitated and then detection is made at a detector for elements to be determined. In so doing, a selector valve is provided along the line for the carrier to replace the carrier with a sample to be detected at an appropriate time.

FIG. 2 is a chart of absorbance detected under such conditions. The absorptiometry for the carrier is represented as a blank value. In contrast, the sample to be detected (the sample) is represented by Δ from the blank value so that a differential may be observed in the absorbance characteristics. The differential Δ is, hence, the difference in absorbance due to the differential between the concentration of the element to be detected, contained in the carrier (presumably, 0) and the concentration of the element to be detected, contained in the sample. Typically, Δ is so small that a procedure for improving the analytical precision is adopted by magnifying Δ by 100 to 1,000 times. Also, fluorescence may be determined in stead of absorbance, for which a fluorescent reagent is used instead of a coupler.

Also in FIA, a differential in absorbance between a carrier and a sample may be amplified by means of an electrical procedure, thereby to increase the analytical sensitivity. To this end, reaction systems or apparatuses having low noises for providing a stable background must be implemented.

Patent Reference 1: Japanese Unexamined Patent Publication No. 2004-163191

Patent Reference 2: Japanese Unexamined Patent Publication No. 1986-108964

Patent Reference 3: Japanese Unexamined Patent Publication No. 1991-235019

### DISCLOSURE OF THE INVENTION

### Problems to be Solved by the Invention

By the use of a liquid chemical bag, FIA may be converted into a completely closed determination system to shut off any contamination from the environment of determination. In addition, they can provide instantaneous results after determination and, moreover, can be easily carried and simply adjusted, which makes them applicable for on-site analyses. As such, they have the advantage that they can be installed in a process of manufacturing semiconductors and the results may immediately be reflected in such a process. Further, since samples are collected at certain time intervals from a chemical to be analyzed (for example, sulfuric acid, nitric acid, hydrochloric acid, hydrofluoric acid, phosphoric acid, aqueous ammonia or aqueous hydrogen peroxide) and such samples are pretreated before absorbance determination with the use of a coupler, metals can be detected even if the samples are strongly acidic or strongly alkaline. Also, since all the steps can be carried out inline according to this method, metallic elements can be confirmed in real-time.

According to the conventional FIA instruments or methods of determination, however, if an exothermic reaction occurs during pretreatment (for example, neutralization) as described above, the pretreated solution (treated solution) may develop foaming phenomenon, so that determination may be unfeasible. To cope with this, the concentration of a sample is reduced at the time of pretreatment; in such a case, however, a new problem arises in which a decrease in sensitivity may be incurred due to an increase in dilution ratio of the sample.

Therefore, the present invention has a principal object of enabling ultratrace analytes that are heretofore undeterminable at a high sensitivity to be determined, by preventing the foaming phenomenon described above. Further, the present invention has preferably an object of providing a means for increasing the detection sensitivity for impurities in a sample from the conventional ppb order to sub-ppb or ppt order by lowering a dilution ratio of the sample.

Patent Reference 2 discloses a method of quantitatively determining trace calcium in an aqueous solution and, in particular, a technique of applying a masking reagent to a sample liquid for masking calcium as an element to be detected. Disclosed as masking reagents to be used therefor are typical chelating agents used as titration reagents for chelating titrations, such as ethylenediamine tetraacetate, ethylene glycol bis(2-aminoethyl)etherdiamine tetraacetate, diethylenetriamine pentaacetate, triethylenetetramine hexaacetate and other salts. It is described therein that, according to this invention, since a comparison is made between a blank agent of a sample liquid to which a masking agent is added and the sample liquid, the both liquids have the common background so that errors resulting from liquidity of the sample liquid may be compensated for. This invention is, however, not directed to providing an ultrahigh purity analysis, because no disclosures are made of application of the technique to FIA.

In addition, Patent Reference 3 discloses an example of using an anionic exchange resin for a purification column for carrier liquid for sample solution and a chelating resin for a purification column for carrier liquid for reagent solution, in order to lower the concentrations of impurities contained in the carrier liquids for the purpose of increasing the analytical sensitivity. In this example, enrichment columns are used in conjunction. In such a method, impurities will elute from a column filler or an eluent which is used after concentration and, for ultratrace analyses, the concentrations of such eluted impurities may sometimes exceed the concentrations of impurities contained in a sample to be detected, therefore, preventing this method from being applied to ultratrace analyses.

### Means for Solving the Problems

The present invention (1) is a method of real-time/inline detection of an ultratrace metallic element contained in a sample liquid, comprising a sampling step of sampling from the sample liquid, a pretreatment step of treating the sample with a pretreatment liquid, and an analyzing step of analyzing the metallic element in a treated liquid that has passed through the pretreatment step, wherein the sample, the pretreatment liquid and/or the treated liquid are cooled, if an exothermic reaction is involved in the pretreatment step.

The present invention (2) is the method according to the invention (1) wherein the pretreatment step is carried out in a plurality of stages.

The present invention (3) is the method according to the invention (1) or (2) wherein the sample liquid is an acid or alkali.

The present invention (4) is the method according to the invention (3) wherein the acid is sulfuric acid, nitric acid, hydrochloric acid, hydrofluoric acid, phosphoric acid or a mixed acid comprising one or more of these acids and the alkali is aqueous ammonia or tetramethyl ammonium hydroxide.

The present invention (5) is the method according to the invention (3) or (4) wherein the acid or the alkali contained in the sample liquid has a concentration of 10 N or more.

The present invention (6) is the method according to any one of the inventions (1) to (5) wherein the metallic element is an element of Groups 6 to 12 of the periodic table.

The present invention (7) is the method according to invention (6) wherein the metallic element is iron (Fe) or copper (Cu).

The present invention (8) is the method according to any one of the inventions (1) to (7) wherein the sample and/or the pretreatment liquid are degassed in advance.

The present invention (9) is the method according to the invention (8) wherein the sample and/or the pretreatment liquid degassed in advance are contained in a highly airtight container.

The present invention (10) is a method of real-time/inline detection of an ultratrace metallic element contained in a sample liquid, comprising a sampling step of sampling from the sample liquid, a pretreatment step of treating the sample with a pretreatment liquid, and an analyzing step of analyzing the metallic element in a treated liquid that has passed through the pretreatment step, wherein the sample and/or the pretreatment liquid are degassed in advance, if an exothermic reaction is involved in the pretreatment step.

The present invention (11) is the method according to the invention (10) wherein the sample and/or the pretreatment liquid degassed in advance are contained in a highly airtight container.

The present invention (12) is the method according to the invention (10) or (11) wherein the pretreatment step is carried out in a plurality of stages.

The present invention (13) is the method according to any one of the inventions (10) to (12) wherein the sample liquid is an acid or alkali.

The present invention (14) is the method according to the invention (13) wherein the acid is sulfuric acid, nitric acid, hydrochloric acid, hydrofluoric acid, phosphoric acid or a mixed acid comprising one or more of these acids and the alkali is aqueous ammonia or tetramethyl ammonium hydroxide.

The present invention (15) is the method according to the invention (13) or (14) wherein the acid or the alkali contained in the sample liquid has a concentration of 10 N or more.

The present invention (16) is the method according to any one of the inventions (10) to (15) wherein the metallic element is an element of Groups 6 to 12 of the periodic table.

The present invention (17) is the method according to Claim (16) wherein the metallic element is iron (Fe) or copper (Cu).

The present invention (18) is an apparatus capable of carrying out real-time/inline detection of an ultratrace metallic element contained in a sample liquid, comprising a sampling section for sampling from the sample liquid, a pretreatment section for treating the sample with a pretreatment liquid, and an analyzing section for analyzing the metallic element in a treated liquid from the pretreatment section, wherein the apparatus further includes a means for cooling the sample, the pretreatment liquid and/or the treated liquid, if an exothermic reaction is involved in treatment with the pretreatment liquid.

The present invention (19) is the apparatus according to the invention (18) which has a plurality of the pretreatment sections and is configured to be capable of carrying out the pretreatment in a plurality of stages.

The present invention (20) is the apparatus according to the invention (18) or (19) wherein the sample liquid is an acid or alkali.

The present invention (21) is the apparatus according to the invention (20) wherein the acid is sulfuric acid, nitric acid, hydrochloric acid, hydrofluoric acid, phosphoric acid or a mixed acid comprising one or more of these acids and the alkali is aqueous ammonia or tetramethyl ammonium hydroxide.

The present invention (22) is the apparatus according to the invention (20) or (21) wherein the acid or the alkali contained in the sample liquid has a concentration of 10 N or more.

The present invention (23) is the apparatus according to any one of the inventions (18) to (22) wherein the metallic element is an element of Groups 6 to 12 of the periodic table.

The present invention (24) is the apparatus according to invention (23) wherein the metallic element is iron (Fe) or copper (Cu).

The present invention (25) is the apparatus according to any one of the inventions (18) to (24) which can be fitted with a highly airtight container in which the sample and/or the pretreatment liquid degassed in advance are contained.

The present invention (26) is a highly airtight container adapted for use with an apparatus capable of carrying out real-time/inline detection of an ultratrace metallic element contained in a sample liquid, the apparatus comprising a sampling section for sampling from the sample liquid, a pretreatment section for treating the sample with a pretreatment liquid, and an analyzing section for analyzing the metallic element in a treated liquid from the pretreatment section, wherein an exothermic reaction may be involved in treatment with the pretreatment liquid, in which the sample and/or the pretreatment liquid degassed in advance are contained.

The present invention (27) is the highly airtight container according to the invention (26) wherein the apparatus further includes a cooling means for cooling the sample, the pretreatment liquid and/or the treated liquid.

The present invention (28) is the highly airtight container according to the invention (27) wherein the apparatus has a plurality of the pretreatment sections and is configured to be capable of carrying out the pretreatment in a plurality of stages.

The present invention (29) is the highly airtight container according to any one of the inventions (26) to (28) wherein the sample liquid is an acid or alkali.

The present invention (30) is the highly airtight container according to the invention (29) wherein the acid is sulfuric acid, nitric acid, hydrochloric acid, hydrofluoric acid, phosphoric acid or a mixed acid comprising one or more of these acids and the alkali is aqueous ammonia or tetramethyl ammonium hydroxide.

The present invention (31) is the highly airtight container according to the invention (29) or (30) wherein the acid or the alkali contained in the sample liquid has a concentration of 10 N or more.

The present invention (32) is the highly airtight container according to any one of the inventions (26) to (31) wherein the metallic element is an element of Groups 6 to 12 of the periodic table.

The present invention (33) is the highly airtight container according to invention (32) wherein the metallic element is iron (Fe) or copper (Cu).

Terms as used herein will now be defined with respect to their meanings. The term “sample liquid” is not particularly limited and includes any liquids treated at a plant, such as a cleaning liquid used in a process for manufacturing semiconductors (for example, an acid or alkali, including such containing hydrogen peroxide added) . The term “real-time” means that, in a situation where a desired product is being produced (treated) using a sample liquid in the plant, a sample liquid that is substantially identical to the sample liquid used is simultaneously analyzed. The term “inline” means that analyses are carried out within the plant. The term “detection” means a quantitative analysis of a metallic element (including a quantitative analysis to such an extent that it may be an index for indicating that the metallic element is present at or below a predetermined value, instead of a precise quantitative determination). A means for “detection” is not particularly limited and includes an absorptiometer and ICP-MS, for example. The term “ultratrace” refers to a concentration for analyses at sub-ppb or ppt order or, preferably, below 1 ppt. The term “cooling” is not particularly limited, as long as it lowers the temperature of a system at a pretreatment step.

Preferably, it means cooling to such an extent that foaming due to an exothermic reaction may substantially be eliminated. The term “a plurality of stages” means two or more stages, with no upper limits specified, but usually, five or less stages in consideration of effects, costs and the like. The term “mixed acid” is not particularly limited as long as it is an acidic mixed liquid containing at least one selected from sulfuric lo acid, nitric acid, hydrochloric acid, hydrofluoric acid and phosphoric acid, and also encompasses a mixed acid containing any of the inorganic acids specifically mentioned above in combination with another acid (for example, an organic acid), such as one of sulfuric acid and acetic acid. The term “highly airtight container” is not particularly limited as long as it is a container having airtightness to such an extent that outside air may not have a substantial influence on a degassed sample and/or treated liquid. Preferably, a container having an internal oxygen permeability of 2.0 fmol/(m²·s·Pa) or less may be mentioned. The term “cooling means for cooling the sample, the pretreatment liquid and/or the treated liquid” may be one that cools each of the objects to be cooled (the sample, the pretreatment liquid and the treated liquid) independently (independently in a physical manner), or one that cools them in groups each consisting of some of them, or one that cools them altogether.

### Effect of the Invention

According to the present invention, since foaming of a liquid that has been pretreated (treated liquid) can be prevented, such an effect is obtained that analyses based on new combinations of samples and treated liquids, which have not been possible heretofore with real-time/inline analyses, may be enabled. In addition, since a concentration of a pretreatment liquid can be increased, such an effect is obtained that the detection sensitivity for impurities in a sample may be increased to sub-ppb or ppt order.

### BEST MODE FOR CARRYING OUT THE INVENTION

Best modes for the present invention will be described below with reference to the drawings. The technical scope of the present invention is not limited to the best modes as described below. In other words, the best modes are only illustrative and any modes having substantially identical constitution and similar effects to the technical ideas described in Claims shall be covered by the technical scope of the present invention.

First, with reference to FIGS. 3 and 4, a first best mode of the present invention will be described in detail. FIG. 3 is a flowchart explaining steps for relevant FIA. As shown in FIG. 3, the FIA includes a sampling step (Step S1) of sampling continually or at a certain time interval from a chemical to be analyzed such as a semiconductor step; a pretreatment step (Step S2) of treating the sample collected at the sampling step with a pretreatment liquid; and an analyzing step (Step S3) of analyzing the metallic element in a treated liquid that has passed through the pretreatment step. Each of these steps will be described in detail below.

(1) Sampling Step

Sampling step S1 is a step of sampling from a sample liquid as a solution to be detected. The sampling should preferably be made at a certain time interval and, more preferably, be made in a certain amount at a certain time interval. There are no particular limitations as to specific methods for sampling.

(2) Pretreatment Step

Pretreatment step S2 is a step of pretreating the sample by injecting (adding) a pretreatment liquid into the collected sample. The pretreatment step is not also particularly limited as long as it involves an exothermic reaction and includes neutralization, dilution and oxidation/reduction treatments, for example. The pretreatment liquid is not particularly limited and can appropriately be selected according to the type of sample liquids as solutions to be detected. For example, in case where neutralization treatment is used as a pretreatment step, when a solution to be detected is one of hydrochloric acid, aqueous ammonia and sodium hydroxide may preferably be used and, when a solution to be detected is one of potassium hydroxide, hydrochloric acid, acetic acid and the like may preferably be used.

(3) Analyzing Step

Analyzing step S3 is a step of analyzing the metallic element in a treated liquid that has passed through the pretreatment step. Methods for analyzing are not particularly limited and include absorptiometry and fluorometry, for example. Preferably, absorptiometry may be mentioned in which colors are produced in a sample treated with a pretreatment liquid by undergoing an oxidative reaction catalyzed by a metal element to be detected and the colors are determined. In this case, N,N-dimethyl-p-phenylenediamine may preferably be used as a coupler and, when N,N-dimethyl-p-phenylenediamine is used, hydrogen peroxide may preferably be used as an oxidizer.

With reference to FIG. 4, this best mode will be described in more detail. FIG. 4 is a schematic drawing of a first embodiment of a detection apparatus according to the present invention. This detection apparatus is a type of flow injection analyzer in which a sample 1 collected from a sample liquid in a certain amount flows into a sample flow tube 2. A pretreatment liquid 3 passes through a pretreatment flow tube 4. The sample 1 and the pretreatment liquid 3 react while passing through a pretreatment tube 5 to make a liquid as pretreated (treated liquid) 16. The liquid as pretreated 16 is heated by exothermic heat during the pretreatment and is however cooled at a cooler 6. Therefore, a temperature rise during the pretreatment may be suppressed and foaming of the liquid as pretreated 16 may be prevented.

The pretreatment tube 5 is connected to an automatic selector valve 210, which is provided with a sample holding tube 101. The sample holding tube 101 is connected to a carrier liquid flow tube 102, so that by switching the automatic selector valve 210 at appropriate timing while a carrier liquid 201 hermetically sealed in a chemicals bag 110 flows into the carrier liquid flow tube 102, the carrier liquid 201 may flow into the sample holding tube 101. Consequently, the sample held in the sample holding tube 101 is forced out into the carrier liquid 201 to be fed into a reaction tube 106.

Connected upstream the reaction tube 106 are a coupler liquid flow tube 104 for feeding a coupler liquid 203, said liquid producing colors by undergoing an oxidative reaction catalyzed by a metal ion; an oxidizer liquid flow tube 103 for feeding an oxidizer liquid 202; and a buffer liquid flow tube 105 for feeding a buffer liquid 204. The liquid as pretreated 16, the coupler liquid 203, the oxidizer liquid 202 and the buffer liquid 204 are mixed together to promote the reaction. The reaction tube 106 can be adjusted in temperature by a temperature regulator 107. By adjustment in temperature of the reaction tube, the rate of color producing reactions by a metal ion can be adjusted.

The reaction tube 106 is connected to an absorptiometer 108 where absorbance that corresponds to the amount of metal ions as impurities in the sample 1 is determined. The sample 1, of which absorbance has been determined, is discharged through a discharge tube 109.

FIG. 5 is a schematic drawing of a pretreatment section of a second best mode of the detection apparatus according to the present invention. A sample 1 collected in a certain amount from a sample liquid 100 flows into a sample flow tube 2. The sample 1 is cooled by a cooler 9 while passing through the sample flow tube 2. In addition, a pretreatment liquid 3 cooled at a pretreatment liquid refrigerator 8 flows into a pretreatment liquid flow tube 4. The sample 1 and the pretreatment liquid 3 react while passing through a pretreatment tube 5 to make a liquid as pretreated 16. The liquid as pretreated 16 is heated by exothermic heat during the pretreatment; however, since both the sample 1 and the pretreatment liquid 3 have been cooled at the cooler and the refrigerator, respectively, a temperature rise during the reaction may be suppressed and foaming of the liquid as pretreated 16 may be prevented in comparison with the case of the first embodiment. Consequently, the pretreatment liquid may be used in a higher concentration in comparison with the case of the first embodiment so that the detection sensitivity for metal ions in the sample may be increased.

FIG. 6 is another schematic drawing of the pretreatment section of the second embodiment. A pretreatment liquid cooler 10 is provided in substitution for the pretreatment liquid refrigerator 8. Here too, the pretreatment liquid may be used in a higher concentration in comparison with the case of the first embodiment so that the detection sensitivity for metal ions in the sample may be increased.

FIG. 7 is a schematic drawing of an embodiment of a third pretreatment section of the detection apparatus. A sample 1 collected in a certain amount from a sample liquid 100 flows into a sample A flow tube 24. The sample 1 is cooled by a cooler 28 while passing through the sample A flow tube 24. In addition, a pretreatment liquid 3 cooled at a pretreatment liquid refrigerator 8 flows into a pretreatment liquid flow tube 4. The sample 1 and the pretreatment liquid 3 react while passing through a pretreatment tube A 25 to make a liquid as pretreated A 17. On the other hand, the sample 1 cooled at a sample B cooler 13 flows into a sample B flow tube 11 and reacts with the liquid as pretreated A 17 while passing through a pretreatment tube B 14 to make a liquid as pretreated B 18. The liquid as pretreated A 17 is heated by exothermic heat during the pretreatment; however, since both the sample 1 and the pretreatment liquid 3 are beforehand cooled at the cooler and the refrigerator, respectively, a temperature rise during the reaction may be suppressed and foaming may be prevented. Further, by cooling the liquid as pretreated A 17 and the sample 1, a temperature rise during the reaction of the liquid after treatment B 18 may be suppressed and foaming may be prevented. Thus, by carrying out the pretreatment in a plurality of stages with cooling for each stage, a temperature rise during the pretreatment may be suppressed. Consequently, the pretreatment liquid may be increased in concentration in comparison with the case of the second embodiment so that the detection sensitivity for metal ions in the sample may be increased.

In the best modes, it is preferred that an environment is established where minimum air may be allowed into the sample and/or the pretreatment liquid. When the sample or the pretreatment liquid is cooled, solubility for air will increase. Increased solubility allows more foaming to occur during the pretreatment step. By containing the sample and the pretreatment liquid in a highly airtight container, however, the air may be shut off so that foaming may be suppressed. Further, by degassing beforehand the sample and/or the pretreatment liquid by, for example, providing the apparatus with a means for degassing, foaming may further be suppressed. In so doing, a highly airtight container having an oxygen permeability of 2.0 fmol/(m²·s·Pa) or less may preferably be used, although any containers capable of shutting off the air may be effective. Thus, by degassing the sample and/or the pretreatment liquid and by also shutting off the air, the pretreatment liquid may be increased in concentration so that the detection sensitivity for metal ions in the sample may further be increased.

### EXAMPLES

With reference to FIG. 4, the apparatus for the example will be described. For pumping of a sample 1 and a pretreatment liquid 3, a Cavro XL 3000 Modular Digital Pump manufactured by Carvo Scientific Instruments, Inc. is used. A pretreatment tube 5 has an internal diameter of 1 mm and a length of 10 cm. A water-cooled cooler 6 is equipped. A six-way valve is equipped for the selection of samples. For pumping of a carrier liquid 201, an oxidizer liquid 202, a coupler liquid 203 and a buffer liquid 204, an APZ-2000 Double Plunger Pump manufactured by Asahi Techneion Co., Ltd. is equipped. This pump is capable of pumping at 0.1 ml/min to 1.4 ml/min. A reaction tube 106 has an internal diameter of 0.8 mm and a length of 200 cm. A temperature regulator capable of regulation from the room temperature to 50° C. is equipped. An absorptiometer capable of measuring wavelengths of 500 to 560 nm is equipped.

### Example 1

In Example 1, occurrence of exothermic heat and foaming in a pretreatment step was examined. The method for pretreatment is described with reference to FIG. 4. In this example, 97% sulfuric acid containing 0.9 ppb of iron was used as the sample 1. Aqueous ammonia was used as the pretreatment liquid 3. These two liquids were pretreated using a Cavro XL 3000 Modular Digital Pump manufactured by Carvo Scientific Instruments, Inc. to determine iron in the sulfuric acid. The sample flow tube 2 and the pretreatment liquid flow tube 4 were cooled with ice-water (0° C.). A tube having an internal diameter of 1 mm and a length of 10 cm was used as the pretreatment tube 5. Also the pretreatment liquid was contained in a highly airtight container and degassed before use.

### Condition (1)

Sample: 97% (18.2 mol/l) sulfuric acid
Amount of sample: 300 μl
Pretreatment liquid: 2.85% (1.65 mol/l) aqueous ammonia
Amount of pretreatment liquid: 5500 μl
Period of time for sample and pretreatment liquid extrusion: 6 min
Flow rate of sample extrusion: 50 μl/min
Flow rate of pretreatment liquid extrusion: 916.7 μl/min
Specification 1: Sample and pretreatment liquid not cooled. Liquid as pretreated not cooled.
Specification 2: Sample and pretreatment liquid cooled. Liquid as pretreated not cooled.
Specification 3: Sample and pretreatment liquid cooled. Liquid as pretreated cooled.

### Table 1

TABLE 1
Results of Experiment under Condition (1)

| | liquid temperature | |
|---|---|---|
| | after pretreatment (° C.) | occurrence of foaming |
| Spec. 1 | 45 | not found |
| Spec. 2 | 37 | not found |
| Spec. 3 | 21 | not found |

### Condition (2)

Sample: 97% (18.2 mol/l) sulfuric acid
Amount of sample: 505 μl
Pretreatment liquid: 4.85% (2.78 mol/l) aqueous ammonia
Amount of pretreatment liquid: 5500 μl
Period of time for sample and pretreatment liquid extrusion: 6 min
Flow rate of sample extrusion: 84 μl/min
Flow rate of pretreatment liquid extrusion: 916.7 μl/min
Specification 1: Sample and pretreatment liquid not cooled. Liquid as pretreated not cooled.
Specification 2: Sample and pretreatment liquid cooled. Liquid as pretreated not cooled.
Specification 3: Sample and pretreatment liquid cooled. Liquid as pretreated cooled.

### Table 2

TABLE 2
Results of Experiment under Condition (2)

| | liquid temperature | |
|---|---|---|
| | after pretreatment (° C.) | occurrence of foaming |
| Spec. 1 | 63 | found |
| Spec. 2 | 52 | not found |
| Spec. 3 | 25 | not found |

Based on the results of determination, no foaming was found under the condition (1) where the sample was set low in concentration with or without cooling. On the other hand, under the condition (2) where the sample was set higher in concentration, foaming phenomenon was observed with Specification 1 without cooling while no foaming phenomenon was observed with Specifications 2 and 3 with cooling. In addition, exothermic temperatures were kept low with cooling.

### Example 2

In Example 2, the pretreatment step was carried out in a plurality of stages. Conditions and results of determination are shown below.

### Condition (3)

Sample: 97% (18.2 mol/l) sulfuric acid
Amount of sample: 846 μl
Pretreatment liquid: 8.45% (4.66 mol/l) aqueous ammonia
Amount of pretreatment liquid: 5500 μl
Period of time for sample and pretreatment liquid extrusion: 6 min

First, the whole sample 846 μμl was divided into Sample A 400 μl and Sample B 446 μl. Sample A 400 μl was mixed with the pretreatment liquid 5500 ρl to produce a liquid as pretreated A. Sample A and the pretreatment liquid were kept at 15° C. Mixing the two liquids would have generated heat; however, the temperature of the liquid as pretreated A was kept at 15° C. by cooling. Next, the liquid as pretreated A 5900 μl was mixed with Sample B 446 μl to produce a liquid as pretreated B. The liquid as pretreated A and Sample B were kept at 15° C. Mixing the two liquids would have generated heat; however, the temperature of the liquid as pretreated B was kept at 23° C. by cooling. No foaming was found with the liquid as pretreated B. As illustrated in Example 2 above, by cooling the sample and the pretreatment liquid before the pretreatment step, by cooling the liquid as pretreated and by also carrying out the pretreatment in a plurality of stages, foaming was successfully prevented with no decrease in concentration of the pretreatment liquid. As a result, the pretreatment liquid was increased in concentration.

### Example 3

In Example 3, the liquid as pretreated 16 that was treated in the pretreatment steps in Examples 1 and 2 was determined with a flow injection analyzer. The method of this example will be described with reference to FIG. 4. For pumping of a carrier liquid 201, an oxidizer liquid 202, a coupler liquid 203 and a buffer liquid 204, an APZ-2000 Double Plunger Pump manufactured by Asahi Techneion Co., Ltd. was used. As the carrier liquid 201, aqueous ammonium sulfate solution was used and fed at a flow rate of 0.8 ml/min. As the oxidizer liquid 202, 0.3% aqueous hydrogen peroxide was used and fed at a flow rate of 0.8 ml/min. As the coupler liquid 203, 4 mmol/l N,N-dimethyl-p-phenylenediamine was used and fed at a flow rate of 0.5 ml/min. As the buffer liquid 204, 1.3 mol/l ammonium acetate was used and fed at a flow rate of 0.5 ml/min. As a sample holding tube 101, a tube having an inner diameter of 0.8 mm and a length of 160 cm was used. The liquid as pretreated 16, the oxidizer liquid 202, the coupler liquid 203 and the buffer liquid 204 were mixed in a reaction tube 106 having an inner diameter of 0.8 mm and a length of 200 cm. The mixed liquid was kept at 35° C. in a temperature regulator 107. The absorbance of this colored solution was determined with a detector at a maximum absorptive wavelength of 514 nm. A tube having an inner diameter of 0.8 mm was used to form the channel. As a carrier liquid, ammonium sulfate at various concentrations depending on each determination was used.

Flow signals for iron in concentrated sulfuric acid are shown in FIG. 8. The condition (1), condition (2) and condition (3) in FIG. 8 correspond to the conditions (1), (2) and (3) described above. The liquid as pretreated 16 under the condition (1) was determined with a flow injection analyzer to find an absorbance of 0.0025. The liquid as pretreated 16 under the condition (2) was determined to find an absorbance of 0.0090, achieving a 3.6 times increase in sensitivity over that under the condition (1). The liquid as pretreated B 18 under the condition (3) was determined to find an absorbance of 0.0156, achieving a 6.2 times increase in sensitivity over that under the condition (1). Thus, the detection sensitivity for impurities in the sample was improved by the increase in concentration of the pretreatment liquid.

Illustration was made in BEST MODE and EXAMPLES herein on the premise of absorptiometry using color developing reactions as a method for detection; however, the present invention is not limited thereto. For example, use of fluorometry, atomic absorption photometry, ICP spectrometry, ICP mass spectrometry and the like as methods for detection may provide similar effects.

### BRIEF DESCRIPTION OF THE DRAWINGS

FIG. 1 is a schematic drawing of a measurement principle according to the present invention;

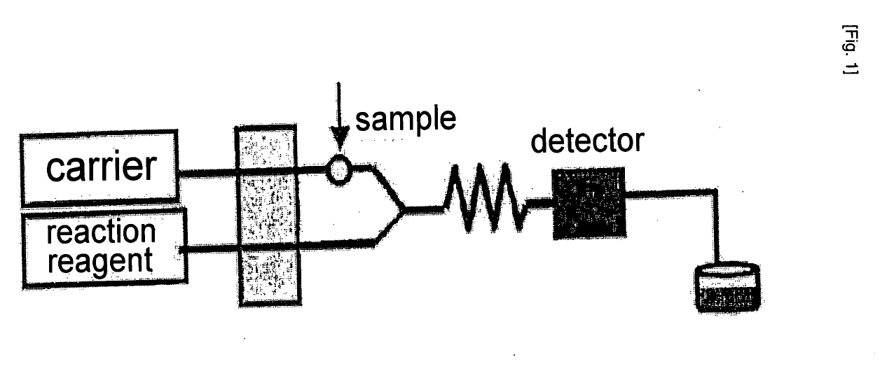

FIG. 2 is a schematic drawing of a measurement principle according to the present invention;

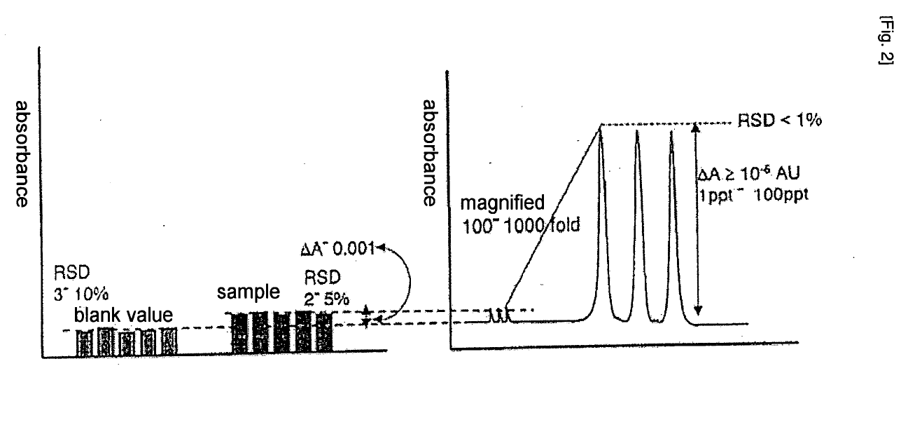

FIG. 3 is a flow chart of determination steps according to the present invention;

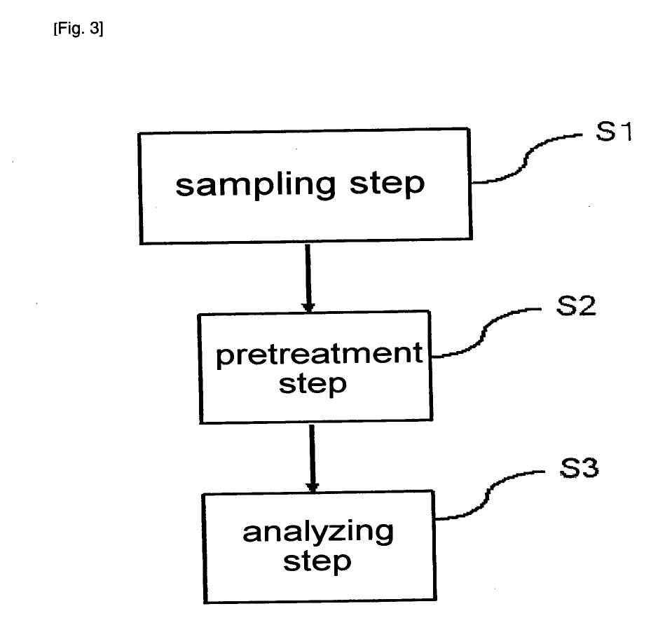

FIG. 4 is a schematic drawing of an apparatus to be used for the present invention;

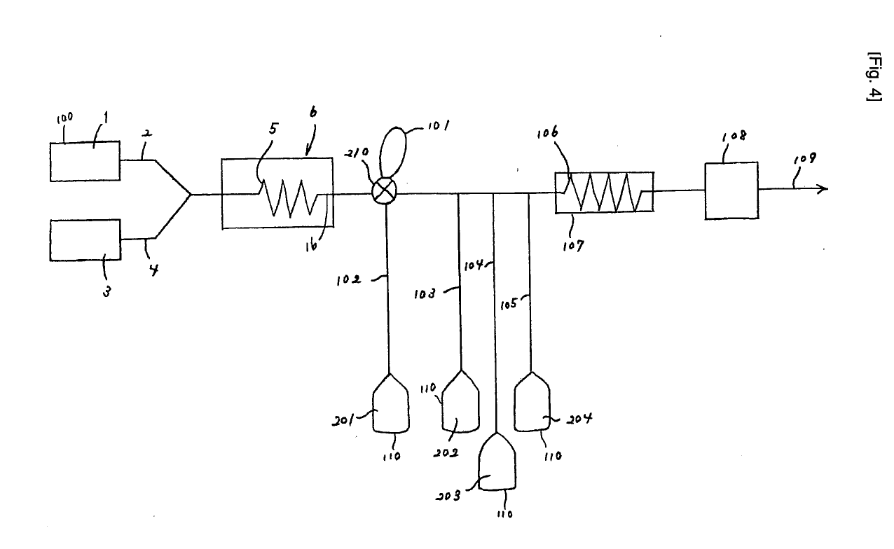

FIG. 5 is a schematic drawing of an apparatus to be used for the present invention;

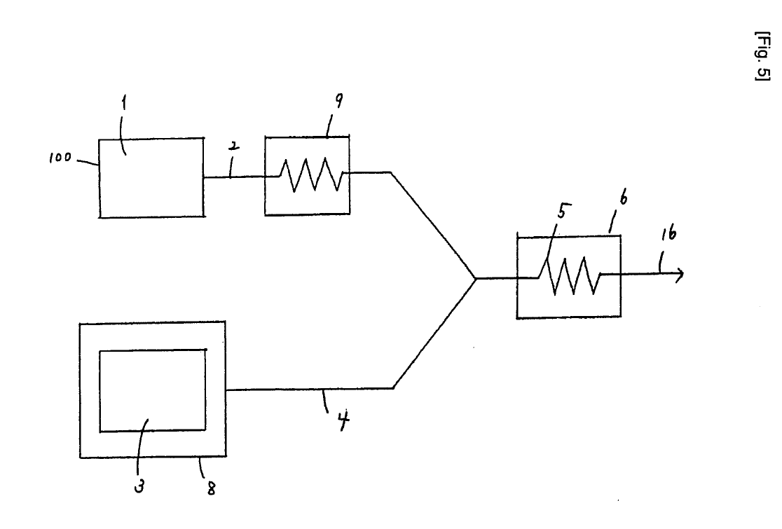

FIG. 6 is a schematic drawing of an apparatus to be used for the present invention;

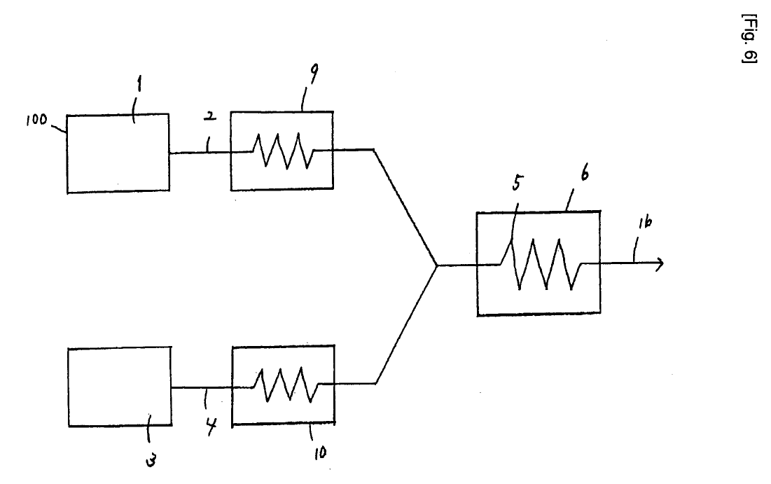

FIG. 7 is a schematic drawing of an apparatus to be used for the present invention; and

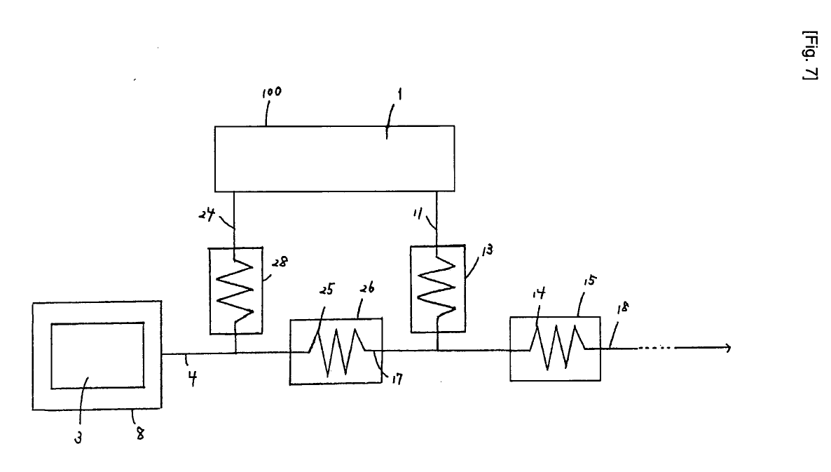

FIG. 8 is a data diagram for determination of trace iron in concentrated sulfuric acid using the present invention.

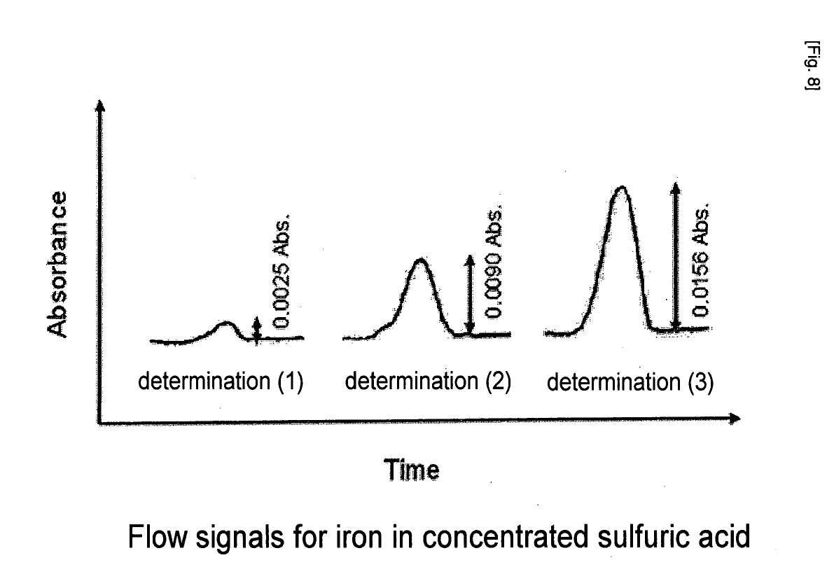

### DESIGNATION OF REFERENCE NUMERALS

1: sample
2: sample flow tube
3: pretreatment liquid
4: pretreatment liquid flow tube
5: pretreatment tube
6: cooler
8: pretreatment liquid refrigerator
9: sample cooler
10: pretreatment liquid cooler
11: sample B flow tube
13: sample B cooler
14: pretreatment tube B
15: cooling tube B
16: liquid as pretreated
17: liquid as pretreated A
17: liquid as pretreated B
24: sample A flow tube
25: pretreatment tube A
26: cooler A
28: sample A cooler
100: sample liquid
101: sample holding tube
102: carrier liquid flow tube
103: oxidizer liquid flow tube
104: coupler liquid flow tube
105: buffer liquid flow tube
106: reaction tube
107: temperature regulator
108: absorptiometer
109: discharge tube
110: chemicals bag
201: carrier liquid
202: oxidizer liquid
203: coupler liquid
204: buffer liquid
210: automatic selector valve

1. A method of real-time/inline detection of an ultratrace metallic element contained in a sample liquid, comprising a sampling step of sampling from the sample liquid, a pretreatment step of treating the sample with a pretreatment liquid, and an analyzing step of analyzing the metallic element in a treated liquid that has passed through the pretreatment step, wherein the sample, the pretreatment liquid and/or the treated liquid are cooled, if an exothermic reaction is involved in the pretreatment step.

2. The method according to claim 1, wherein the pretreatment step is carried out in a plurality of stages.

3. The method according to claim 1 or 2, wherein the sample liquid is an acid or alkali.

4. The method according to claim 3, wherein the acid is sulfuric acid, nitric acid, hydrochloric acid, hydrofluoric acid, phosphoric acid or a mixed acid comprising one or more of these acids and the alkali is aqueous ammonia or tetramethyl ammonium hydroxide.

5. The method according to claim 3 or 4, wherein the acid or the alkali contained in the sample liquid has a concentration of 10 N or more.

6. The method according to any one of claims 1 to 5, wherein the metallic element is an element of Groups 6 to 12 of the periodic table.

7. The method according to claim 6, wherein the metallic element is iron (Fe) or copper (Cu).

8. The method according to any one of claims 1 to 7, wherein the sample and/or pretreatment liquid are degassed in advance.

9. The method according to claim 8, wherein the sample and/or the pretreatment liquid degassed in advance are contained in a highly airtight container.

10. A method of real-time/inline detection of an ultratrace metallic element contained in a sample liquid, comprising a sampling step of sampling from the sample liquid, a pretreatment step of treating the sample with a pretreatment liquid, and an analyzing step of analyzing the metallic element in a treated liquid that has passed through the pretreatment step, wherein the sample and/or the pretreatment liquid are degassed in advance, if an exothermic reaction is involved in the pretreatment step.

11. The method according to claim 10, wherein the sample and/or the pretreatment liquid degassed in advance are contained in a highly airtight container.

12. The method according to claim 10 or 11, wherein the pretreatment step is carried out in a plurality of stages.

13. The method according to any one of claims 10 to 12, wherein the sample liquid is an acid or alkali.

14. The method according to claim 13, wherein the acid is sulfuric acid, nitric acid, hydrochloric acid, hydrofluoric acid, phosphoric acid or a mixed acid comprising one or more of these acids and the alkali is aqueous ammonia or tetramethyl ammonium hydroxide.

15. The method according to claim 13 or 14, wherein the acid or the alkali contained in the sample liquid has a concentration of 10 N or more.

16. The method according to any one of claims 10 to 15, wherein the metallic element is an element of Groups 6 to 12 of the periodic table.

17. The method according to claim 16, wherein the metallic element is iron (Fe) or copper (Cu).

18. An apparatus capable of real-time/inline detection of an ultratrace metallic element contained in a sample liquid, comprising a sampling section for sampling from the sample liquid, a pretreatment section for treating the sample with a pretreatment liquid, and an analyzing section for analyzing the metallic element in a treated liquid from the pretreatment section, wherein the apparatus further includes a means for cooling the sample, the pretreatment liquid and/or the treated liquid, if an exothermic reaction is involved in treatment with the pretreatment liquid.

19. The apparatus according to claim 18, which has a plurality of the pretreatment sections and is configured to be capable of carrying out the pretreatment in a plurality of stages.

20. The apparatus according to claim 18 or 19, wherein the sample liquid is an acid or alkali.

21. The apparatus according to claim 20, wherein the acid is sulfuric acid, nitric acid, hydrochloric acid, hydrofluoric acid, phosphoric acid or a mixed acid comprising one or more of these acids and the alkali is aqueous ammonia or tetramethyl ammonium hydroxide.

22. The apparatus according to claim 20 or 21, wherein the acid or the alkali contained in the sample liquid has a concentration of 10 N or more.

23. The apparatus according to any one of claims 18 to 22, wherein the metallic element is an element of Groups 6 to 12 of the periodic table.

24. The apparatus according to claim 23, wherein the metallic element is iron (Fe) or copper (Cu).

25. The apparatus according to any one of claims 18 to 24, which can be fitted with a highly airtight container in which the sample and/or the pretreatment liquid degassed in advance are contained.

26. A highly airtight container adapted for use with an apparatus capable of real-time/inline detection of an ultratrace metallic element contained in a sample liquid, the apparatus comprising a sampling section for sampling from the sample liquid, a pretreatment section for treating the sample with a pretreatment liquid, and an analyzing section for analyzing the metallic element in a treated liquid from the pretreatment section, wherein an exothermic reaction may be involved in treatment with the pretreatment liquid, in which the sample and/or the pretreatment liquid degassed in advance are contained.

27. The highly airtight container according to claim 26, wherein the apparatus further includes a cooling means for cooling the sample, the pretreatment liquid and/or the treated liquid.

28. The highly airtight container according to claim 27, wherein the apparatus has a plurality of the pretreatment sections and is configured to be capable of carrying out the pretreatment in a plurality of stages.

29. The highly airtight container according to any one of claims 26 to 28, wherein the sample liquid is an acid or alkali.

30. The highly airtight container according to claim 29, wherein the acid is sulfuric acid, nitric acid, hydrochloric acid, hydrofluoric acid, phosphoric acid or a mixed acid comprising one or more of these acids and the alkali is aqueous ammonia or tetramethyl ammonium hydroxide.

31. The highly airtight container according to claim 29 or 30, wherein the acid or the alkali contained in the sample liquid has a concentration of 10 N or more.

32. The highly airtight container according to any one of claims 26 to 31, wherein the metallic element is an element of Groups 6 to 12 of the periodic table.

33. The highly airtight container according to claim 32, wherein the metallic element is iron (Fe) or copper (Cu).
